(19) United States
(12) Patent Application Publication
(10) Pub. No.: US 2024/0312009 A1
(43) Pub. Date: Sep. 19, 2024

(54) MULTI-SCALE 3D CONVOLUTIONAL CLASSIFICATION MODEL FOR CROSS-SECTIONAL VOLUMETRIC IMAGE RECOGNITION

(71) Applicants: Versitech Limited, Hong Kong (CN); The Education University of Hong Kong, Hong Kong (CN)

(72) Inventors: Keith Wan Hang CHIU, Hong Kong (CN); Wai Kay Walter SETO, Hong Kong (CN); Gilbert Chiu Sing LUI, Hong Kong (CN); Jianliang LU, Hong Kong (CN); Man Fung YUEN, Hong Kong (CN); Philip Leung Ho YU, Hong Kong (CN)

(73) Assignees: Versitech Limited, Hong Kong (CN); The Education University of Hong Kong, Hong Kong (CN)

(21) Appl. No.: 18/576,722

(22) PCT Filed: Jul. 6, 2023
(86) PCT No.: PCT/CN2022/104159

§ 371 Date: Jan. 4, 2024

Related U.S. Application Data
(60) Provisional application No. 63/218,972, filed on Jul. 7, 2021.

(51) Int. Cl. G06T 7/00; G06T 3/40; G06V 10/44; G06V 10/764; G06V 10/80; G16H 50/20

(52) U.S. Cl. CPC G06T 7/0012; G06T 3/40; G06V 10/44; G06V 10/764; G06V 10/806; G16H 50/20; G06T 2207/10081; G06T 2207/10088; G06T 2207/30096; G06V 2201/03

(57) ABSTRACT

A three dimensional classification system for recognizing cross-sectional images automatically contains a processor that executes: (1) rescaling of a plurality of cross-sectional images; and feeding the rescaled plurality of cross-sectional images into two branches; (2) feeding the rescaled plurality of cross-sectional images into a first branch for performing a plurality of convolutions on the rescaled plurality of cross-sectional images directly to learn features for distinguishing phases; (3) feeding the rescaled plurality of cross-sectional images into a second branch for reducing resolution, and then performing a plurality of convolutions on the reduced resolution plurality of cross-sectional images to learn features for distinguishing phases; and (4) concatenating convolutional output channels from the two branches to fuse global and local features, on which two fully-connected layers are stacked as a classifier to recognize cross-sectional volumetric images accurately and quickly.

### CROSS-REFERENCE TO RELATED PATENT APPLICATIONS

This application is a U.S. National Stage Application under 35 U.S.C. § 371 of International Patent Application No. PCT/CN2022/104159, filed Jul. 6, 2022, and claims the benefit of priority under 35 U.S.C. Section 119(e) of U.S. Application No. 63/218,972, filed Jul. 7, 2021, all of which are incorporated herein by reference in their entireties. The International Application was published on Jan. 12, 2023 as International Publication No. WO 2023/280221 A1.

### TECHNICAL FIELD

Disclosed are three dimensional classification systems, methods of recognizing cross-sectional images, and non-transitory machine-readable storage mediums.

### BACKGROUND OF THE INVENTION

Computerized tomography (CT) scan is a crucial medical imaging technique for early cancer diagnosis. CT scans, which include multiple phases, are acquired after injecting radio-opaque contrast media into patients and tracking it in the regions of interest by following standardized protocols for time intervals between intravenous radiocontrast injection and image acquisition. The present invention considers four typical phases: non-contrast phase, arterial phase, portal venous phase and delay phase. FIG. 1 shows comparisons of these four different CT phases for the abdomen of three different patients. Generally, non-contrast phase denotes the phase without contrast media, where CT images are relatively darker, compared to delayed phase. Arterial phase is acquired 35-40 seconds after injecting contrast media, in which structures that obtain blood supply from arteries have optimal enhancement, such as heart and aorta. Venous phase denotes the phase acquired 70-90 seconds after contrast media injection, in which the portal vein and hepatic vein are enhanced. Delayed phase denotes the phase acquired 3-15 minutes after contrast phase media. Experienced radiologists can easily identify the existence of lesions by comparing different phases. However, it is common that there may be some phases missing from the CT scans. Besides, after CT scans have been obtained, the phase information is recorded manually, during which mislabeling may happen inevitably, especially when the cohort is large-scale. Such phase detection and correction is prohibitively resource-intensive. Recent clinical studies that have included qualitative and quantitative evaluations of phased-contrast CT have shown that there are promising potentials for determining lesions and abnormal tissues in certain organs.

Inspired by successes with deep learning in computer vision applications, researchers have utilized the advanced related methods to interpret and analyze diagnostic CT images. In this setting, the adoption of deep neural network based methods can be considered for contrast CT phase classification. For example, contrast phase classification for CT images was proposed by utilizing the powerful capability of Generative Adversarial Networks (GANs). Meanwhile, the effects of the backbones of the discriminator in GANs, which has two roles, were investigated as to the ability to identify contrast CT phase images and to distinguish generated CT phase images from real ones. However, this method is a 2D model and only considers three types of phases, namely, non-contrast phase, portal venous phase and delayed phases. A 3 dimensional squeeze-and-extraction (3DSE) network for CT phase recognition was proposed, in which a squeeze-and-excitation mechanism was introduced for capturing global information. Further an aggregated cross-entropy was proposed for combining CT phase images and weak supervision information of the corresponding text descriptions. A 3D-convolutional network was proposed to capture spatiotemporal features. Enlighted by residual networks, a 3D residual network to learn spatiotemporal features was proposed for action recognition in video. Although the two methods were originally designed to model appearance and motion for video content analysis, they were also suitable for classifying CT phases since there exist a time relationship across phases of CT scans. The effectiveness of these two methods in recognizing CT phases have been proven. However, these methods seldom consider multi-scale information for CT phases, since features learned by convolutions of the same kernel can have receptive fields of different sizes when input images have different scales. Besides, there is a lack of research on modelling interactions across convolution channels in 3D classification model.

### SUMMARY OF THE INVENTION

The following presents a simplified summary of the invention in order to provide a basic understanding of some aspects of the invention. This summary is not an extensive overview of the invention. It is intended to neither identify key or critical elements of the invention nor delineate the scope of the invention. Rather, the sole purpose of this summary is to present some concepts of the invention in a simplified form as a prelude to the more detailed description that is presented hereinafter.

Practically, squeeze-and-extraction (SE) was integrated into a 3D model for CT phase recognition via modelling cross-channel interdependencies in order to mine global information. However, such SE leads to a significant increase in model complexity and computational burden. To address these issues, a multi-scale 3D classification network for CT phase recognition (MS3DCN-ECA) is described herein. Experimental results on CT scans collected and reported herein indicate that MS3DCN-ECA achieves state-of-the-art performance in terms of at least one of sensitivity, PPV, F1-score at the phase level, and the best performance in terms of macro-accuracy and micro-accuracy at the overall level.

Disclosed herein is a three dimensional classification system for recognizing cross-sectional images automatically, which system contains a processor that executes: (1) rescaling a plurality of cross-sectional images and feeding the rescaled plurality of cross-sectional images into two branches; (2) feeding the rescaled plurality of cross-sectional images into a first branch for performing a plurality of convolutions on the rescaled plurality of cross-sectional images directly to learn features for distinguishing phases; (3) feeding the rescaled plurality of cross-sectional images into a second branch for reducing resolution and then performing a plurality of convolutions on the reduced resolution plurality of cross-sectional images to learn features for distinguishing phases; and (4) concatenating convolutional output channels from the two branches to fuse global and local features, on which two fully-connected layers are stacked as a classifier to recognize cross-sectional volumetric images accurately and quickly.

To the accomplishment of the foregoing and related ends, the invention comprises the features hereinafter fully described and particularly pointed out in the claims. The following description and the annexed drawings set forth in detail certain illustrative aspects and implementations of the invention. These are indicative, however, of but a few of the various ways in which the principles of the invention may be employed. Other objects, advantages and novel features of the invention will become apparent from the following detailed description of the invention when considered in conjunction with the drawings.

### BRIEF SUMMARY OF THE DRAWINGS

This patent or application file contains at least one drawing executed in color. Copies of this patent or patent application publication with color drawing(s) will be provided by the Office upon request and payment of the necessary fee.

The foregoing and other objects and advantages of the present invention will become more apparent when considered in connection with the following detailed description and appended drawings in which like designations denote like elements in the various views, and wherein:

FIG. 1 depicts conventional comparisons between phases of CT scans of three slices from different patients: Non-Contrast Phase, Arterial Phase, Portal Venous Phase and Delayed Phase.

FIG. 2 depicts the framework of a multi-scale 3D classification network in accordance with an embodiment of the invention for identifying cross-sectional volumetric images, where σ denotes the sigmoid activation function, and the block in the red dashed-line box denotes a base convolutional module.

FIG. 3 depicts a comparison of CT phases and corresponding feature maps learned by a model network in accordance with an embodiment of the invention, where the parts in the black solid-line boxes denote arteries that have different intensities across phases and the parts in the black dash-line boxes denote veins which are clearly in portal venous phase.

FIG. 4 illustrates a block diagram of an exemplary electronic computing environment that can be implemented in conjunction with one or more aspects of the present invention as described herein.

FIG. 5 depicts a block diagram of an exemplary data communication network that can be operable in conjunction with various aspects of the present invention as described herein.

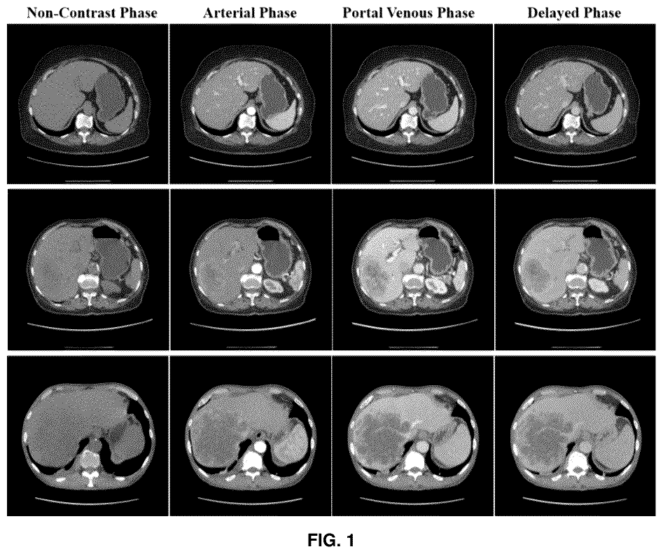

Table 1 reports the detailed classification results obtained by the method of the present invention on the testing data set.

Table 2 reports the comparison results between the method according to the present invention and competing methods in terms of Sensitivity, PPV and F1-Score, where the best results are in bold, and the second best results are in red.

Table 3 reports comparisons between the method of the present invention and competing methods in terms of macro-accuracy and micro-accuracy, where the best results are in bold, and the second best results are in red.

Table 4 reports the comparisons between MS3DCN-ECA and its variants in terms of Sensitivity, PPV and F1-Score. The best results are in bold, and the second best results in red.

Table 5 reports comparison results between the MS3DCN-ECA of the present invention and its variants in terms of micro-accuracy and macro-accuracy, where the best results are in bold, and the second best results are in red.

### DETAILED DESCRIPTION

Nowadays deep learning based methods are used for medical image analysis. However, its implementation is restricted by the availability of large-scale labelled medical images like CT scans, which can be collected from picture archiving and communication systems. With the available CT scans, described herein is a multi-scale 3D classification network (MS3DCN-ECA) for CT phase recognition. Specifically, first the sizes of original CT scans are rescaled from 512 to 256 and the slice number (128) is fixed for reducing the hardware requirement. Then the rescaled CT scans are fed into MS3DCN-ECA which includes two branches. The first branch conducts convolutions on the rescaled imaging (e.g. computed tomography (CT) or magnetic resonance) scans directly, while the second branch further reduced the size from 256 to 128 before convolutions. Considering that channel attention is proven to increase performance gain via modeling cross-channel interdependencies, an efficient channel attention mechanism is introduced to mine inter-correlations across convolutional outputs for each branch. Finally, the information flow from these two branches is flattened and concatenated, followed by the connection of to two fully-connected layers for recognition of cross-sectional volumetric images are stacked.

To demonstrate the effectiveness of MS3DCN-ECA, experiments were conducted and reported on the collected CT scans from multiple centers. MS3DCN-ECA achieves, for example, mean sensitivity of 0.9842, mean PPV of 0.9842, mean F1-score of 0.9840 at the CT phase level. Furthermore, MS3DCN-ECA achieves a macro-accuracy of 0.9841 and micro-accuracy of 0.9920.

The multi-scale 3D convolutional classification model for cross-sectional image recognition (MS3DCN-ECA) described herein, introduces an efficient channel attention mechanism to construct interdependencies among convolutional channels. Since convolutional kernels in the two branches are the same, while the cross-sectional volumetric images of convolution inputs have different resolutions, this multi-scale strategy in the model for the present invention can have receptive fields of different sizes, allowing a flexible fusion of features corresponding to local regions of interest from fine to coarse. Meanwhile, considering that channel attention brings performance gain via modeling cross-channel interdependencies, efficient channel attention mechanism mines inter-correlations across convolutional channels for each branch.

Thus, the present invention is a 3D deep learning network able to capture spatiotemporal features, allowing a quantitative and functional classification of captured visual patterns. This facilitates an assessment of anatomical structures in which a stereoscopic volumetric quantification of its architecture is of clinical relevance. In addition, the unique multi-scale deep learning model of the present invention can recognize and integrate the different phases or sequences of cross-sectional imaging, including computed tomography and magnetic resonance.

Automatically identifying cross-sectional volumetric images and correcting manual recording errors for picture archiving and communication system (PACS) has been a problem. PACS is the universal system currently used in medical imaging.

This problem is addressed by designing a multi-scale 3D convolutional classification network, in which an efficient channel attention mechanism is introduced to model cross-channel interdependencies that capture global information as complementary for convolution. Specifically, the network of the present invention has two branches fed into cross-sectional volumetric images with different resizes which are obtained via rescaling. Each of these two branches is composed of four consecutive convolutional blocks to learn high-level local discriminative features from fine to coarse, with the increase of network depth. Meanwhile, efficient channel attention mechanisms are utilized to model cross-channel interdependencies for capturing global features. Finally, convolutional output channels from the two branches are concatenated to fuse global and local features, on which two fully-connected layers are stacked as a classifier to recognize cross-sectional volumetric images accurately and quickly.

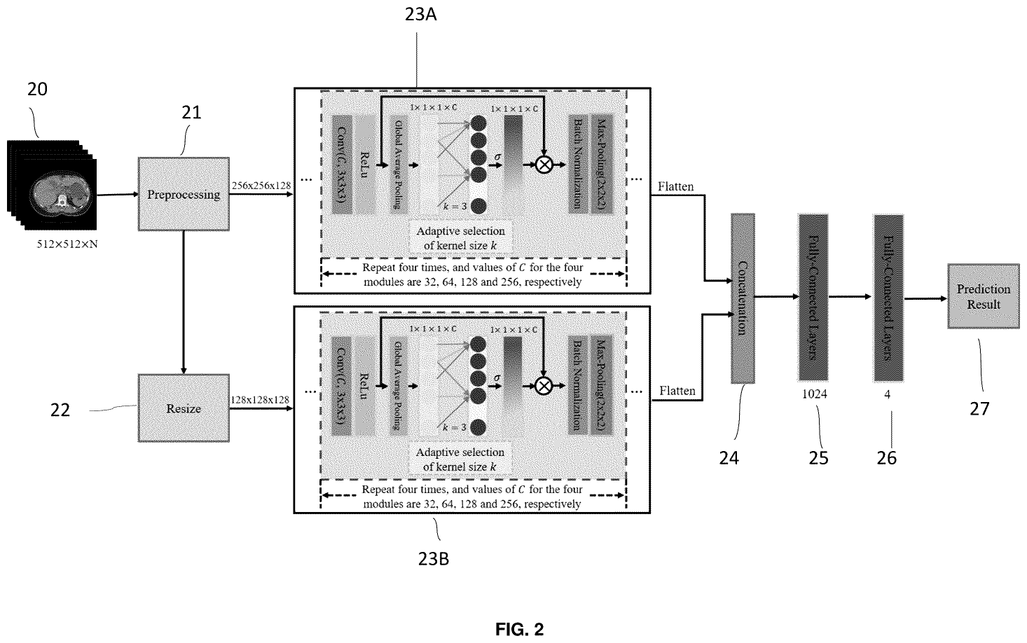

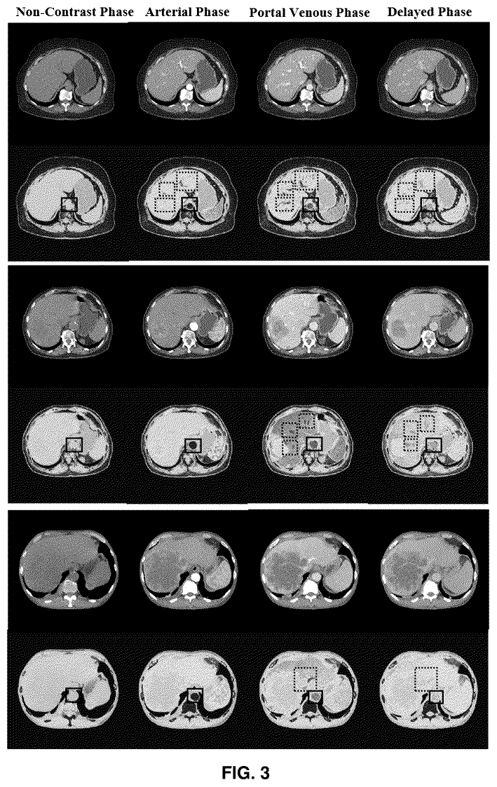

FIG. 2 shows the architecture of the multi-scale 3D classification model of the present invention for recognizing cross-sectional volumetric images automatically. TensorFlow 2.11 was used for the development of the deep learning model of the present invention. This model is composed of two branches A and B, each of which includes four base convolutional modules (where only one each is shown in FIG. 2). The differences in these branches lie in those sizes of their input cross-sectional volumetric images. For image processing, images with different intensities were windowed to the range of [0, 400] for Hounsfield units (HU) and were normalized to [−0.5, 0.5] to remove extraneous features. The raw image size of the CT scans 20 was 512 (height in pixels)×512 (width in pixels)×N, where N denoted the number of slices. Since the number N of different samples are different, there is an image registration step in preprocessing unit 21 to align the number of slice to 128. The input image size of the first branch is (256*256*128) and is resized in unit 22 to (128*128*128) for the second branch.

Conv(C, 3×3×3) is a convolution action in neural networks (23A and 23B) for information extraction in the neural network which creates a convolution kernel that is convolved with the layer input to produce a tensor of outputs. Both “C” and “3×3×3” are the parameters of Conv, named filters and kernel size, respectively. ReLu is the rectified linear activation function, which is a piecewise linear function that will output the input directly if it is positive, otherwise, it will output zero. The Global Average Pooling is GlobalAveragePooling3D action in neural network training. Its output is to the adaptive kernel size selection process. Batch Normalization and Max Pooling are also regular actions in neural network training. In the training processing, the kernel size k is a sensitive parameter and needs to adjust adaptively. For both branches, each of these blocks will repeat 4 times with the filter number of Conv are 32, 64, 128, and 256, respectively.

Since convolutional layers in these two branches have the same-size kernels, 1) receptive fields of the two branches at the same network depth can have different sizes, which increases the richness of semantic features from fine to coarse gradually before flattening and concatenating them later in units 24, 25 and 26; and 2) two of the branches can learn better combinations of feature maps with various scales on key visual cues for distinguishing cross-sectional volumetric images. Meanwhile, efficient channel attention mechanisms are further introduced in each base convolutional module to build interdependencies between feature maps, through which global information at the sectional level can be learned. With the collaboration of multi-scale convolution and efficient channel attention, the network can extract as the prediction result 27 high-level discriminative semantic features beneficial to the recognition of cross-sectional volumetric images successfully. Evaluated on 2714 collected cross-sectional volumetric images, the model achieves a mean sensitivity of 0.9842, mean PPV of 0.9842 and mean F1-score of 0.9840, macro-accuracy of 0.9841 and micro-accuracy of 0.9920. All of these results are much better than conventional methods.

Referring again to FIG. 2, the MS3DCN-ECA for CT phase recognition system relies on visual hints that indicate specific CT phases located in different anatomical slices. For this a 3D classification model that considers spatial relationships across slices is preferable, compared with conventional 2D models. FIG The image size for the upper branch 23A is 256×256, while that for the bottom branch 23B is 128×128. The motivations for adopting two branches with different image sizes are: 1) receptive fields of two branches at the same network depth can have different sizes, which increases the richness of semantic features when concatenating them later; and 2) two branches can learn better combinations of feature maps with various scales on key visual cues for distinguishing CT phases. As a result, feature maps corresponding to key visual cues, in particular anatomical slices learned by these two branches, are stressed and feature maps corresponding to uninterested regions are suppressed.

Since feature maps learned by convolutions are extremely local, it is necessary to inject global information concerning whole slices. Conventionally, squeeze-and-excitation (SE) was used to capture global information, which has brought about an evident performance gain. However, empirical proof indicates that this gain is achieved at the cost of an increase in both model complexity and computational burden. To solve these issues, the efficient channel attention (ECA) in each base convolutional module is introduced to build the interdependencies between feature maps, as shown in the red dash-line box of FIG. 2. Instead of using two fully connected layers with an inverse shape in SE to capture all cross-channel interaction, ECA focuses on local cross-channel interactions only, i.e., each channel and its k-neighbors. Specifically, let the output of a convolutional layer be X∈RH×W×D×C, where H, W, D and C denote height, width, depth and the channel numbers respectively, first conduct global average pooling on X, which leads to a vector y∈RC×1. Then the channel attention can be learned by

$$ω = σ My \qquad ( 1 )$$

where M∈RC×C is the learnable weights for channel attention, and σ is a sigmoid function. Since k neighbors for each convolutional channel are only considered, there are k non-zero items in each row of matrix M. To this end, an efficient yet simple trick is to force all the channels to share the same parameters, which can be easily done via a 1D convolution with kernel size k. Thus, the channel attention can be rewritten as

$$ω = σ Z\_k ( y ) \qquad ( 2 )$$

where Z_k denotes 1D convolution with kernel size k, e.g., k=3.

Finally, feature maps learned by convolutional kernels are multiplied by corresponding channel attention from Eq. (2), achieving the fusion of local and global information.

The new multi-scale 3D convolutional classification network for CT phase recognition, referred to as MS3DCN-ECA, considers multi-scale information fusion learned by two branches that are fed into CT scans of different sizes, where efficient channel attention is used to learn weights for channel attention for capturing global information of those slices, followed by combining local information of key visual cues. A comparative experiment and ablation study was conducted on collected CT scans. The experimental results indicate the model according to the present invention outperforms other competing methods, which indicates its effectiveness and superiority.

### Example Computing Environment

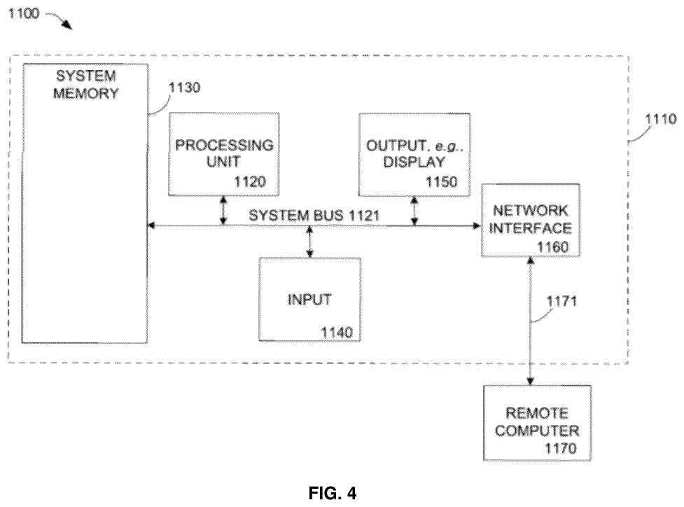

As mentioned, advantageously, the techniques of the present invention can be applied to any device and/or network where analysis of data is performed. The general purpose remote computer described in FIG. 4 is but one example, and the disclosed subject matter can be implemented with any client having network/bus interoperability and interaction. Thus, the disclosed subject matter can be implemented in an environment of networked hosted services in which very little or minimal client resources are implicated, e.g., a networked environment in which the client device serves merely as an interface to the network/bus, such as an object placed in an appliance.

Although not required, some aspects of the disclosed subject matter can partly be implemented via an operating system, for use by a developer of services for a device or object, and/or included within application software that operates in connection with the component(s) of the disclosed subject matter. Software may be described in the general context of computer executable instructions, such as program modules or components, being executed by one or more computer(s), such as projection display devices, viewing devices, or other devices. Those skilled in the art will appreciate that the disclosed subject matter may be practiced with other computer system configurations and protocols.

FIG. 4 thus illustrates an example of a suitable computing system environment 1100 in which some aspects of the disclosed subject matter can be implemented, although as made clear above, the computing system environment 1100 is only one example of a suitable computing environment for a device and is not intended to suggest any limitation as to the scope of use or functionality of the disclosed subject matter. Neither should the computing environment 1100 be interpreted as having any dependency or requirement relating to any one or combination of components illustrated in the exemplary operating environment 1100.

With reference to FIG. 4, an exemplary device for implementing the disclosed subject matter includes a general-purpose computing device in the form of a computer 1110. Components of computer 1110 may include, but are not limited to, a processing unit 1120, a system memory 1130, and a system bus 1121 that couples together various system components including the system memory to the processing unit 1120. The system bus 1121 may be any of several types of bus structures including a memory bus or memory controller, a peripheral bus, and a local bus using any of a variety of bus architectures.

Computer 1110 typically includes a variety of computer readable media. Computer readable media can be any available media that can be accessed by computer 1110. By way of example, and not limitation, computer readable media can comprise computer storage media and communication media. Computer storage media includes nonvolatile, removable and non-removable media implemented in any method or technology for storage of information such as computer readable instructions, data structures, program modules or other data. Computer storage media includes, but is not limited to ROM, EEPROM, flash memory or other memory technology, CDROM, digital versatile disks (DVD) or other optical disk storage, magnetic cassettes, magnetic tape, magnetic disk storage or other magnetic storage devices, or any other medium which can be used to store the desired information and which can be accessed by computer 1110. Communication media typically embodies computer readable instructions, data structures, program modules, or other data in a modulated data signal such as a carrier wave or other transport mechanism and includes any information delivery media.

The system memory 1130 may include computer storage media in the form of nonvolatile memory such as read only memory (ROM). A basic input/output system (BIOS), containing the basic routines that help to transfer information between elements within computer 1110, such as during start-up, may be stored in memory 1130. Memory 1130 typically also contains data and/or program modules that are immediately accessible to and/or presently being operated on by processing unit 1120. By way of example, and not limitation, memory 1130 may also include an operating system, application programs, other program modules, and program data.

The computer 1110 may also include other removable/non-removable, volatile/nonvolatile computer storage media. For example, computer 1110 could include a hard disk drive that reads from or writes to non-removable, nonvolatile magnetic media, a magnetic disk drive that reads from or writes to a removable, nonvolatile magnetic disk, and/or an optical disk drive that reads from or writes to a removable, nonvolatile optical disk, such as a CD-ROM or other optical media. Other removable/non-removable nonvolatile computer storage media that can be used in the exemplary operating environment include, but are not limited to, magnetic tape cassettes, flash memory cards, digital versatile disks, digital video tape, solid state ROM, and the like. A hard disk drive is typically connected to the system bus 1121 through a non-removable memory interface, and a magnetic disk drive or optical disk drive is typically connected to the system bus 1121 by a removable memory interface.

A user can enter commands and information into the computer 1110 through input devices such as a keyboard and pointing device, commonly referred to as a mouse, trackball, or touch pad. Other input devices can include a microphone, joystick, game pad, satellite dish, scanner, wireless device keypad, voice commands, or the like. These and other input devices are often connected to the processing unit 1120 through user input 1140 and associated interface(s) that are coupled to the system bus 1121, but may be connected by other interface and bus structures, such as a parallel port, game port, or a universal serial bus (USB). A graphics subsystem can also be connected to the system bus 1121. A projection unit in a projection display device, or a HUD in a viewing device or other type of display device can also be connected to the system bus 1121 via an interface, such as output interface 1150, which may in turn communicate with video memory. In addition to a monitor, computers can also include other peripheral output devices such as speakers which can be connected through output interface 1150.

The computer 1110 can operate in a networked or distributed environment using logical connections to one or more other remote computer(s), such as remote computer 1170, which can in turn have media capabilities different from device 1110. The remote computer 1170 can be a personal computer, a server, a router, a network PC, a peer device, personal digital assistant (PDA), cell phone, handheld computing device, a projection display device, a viewing device, or other common network node, or any other remote media consumption or transmission device, and may include any or all of the elements described above relative to the computer 1110. The logical connections depicted in FIG. 4 include a network 1171, such as a local area network (LAN) or a wide area network (WAN), but can also include other networks/buses, either wired or wireless. Such networking environments are commonplace in homes, offices, enterprise-wide computer networks, intranets and the Internet.

When used in a LAN networking environment, the computer 1110 can be connected to the LAN 1171 through a network interface or adapter. When used in a WAN networking environment, the computer 1110 can typically include a communications component, such as a modem, or other means for establishing communications over the WAN, such as the Internet. A communications component, such as wireless communications component, a modem and so on, which can be internal or external, can be connected to the system bus 1121 via the user input interface of input 1140, or other appropriate mechanism. In a networked environment, program modules depicted relative to the computer 1110, or portions thereof, can be stored in a remote memory storage device. It will be appreciated that the network connections shown and described are exemplary and other means of establishing a communications link between the computers can be used.

### Exemplary Networking Environment

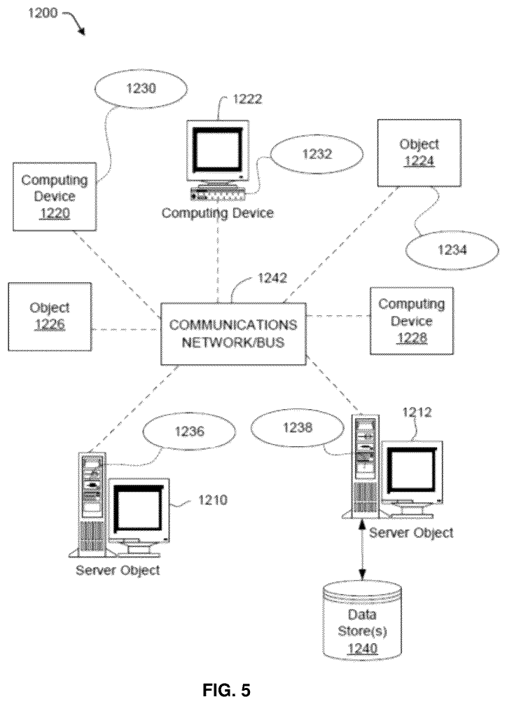

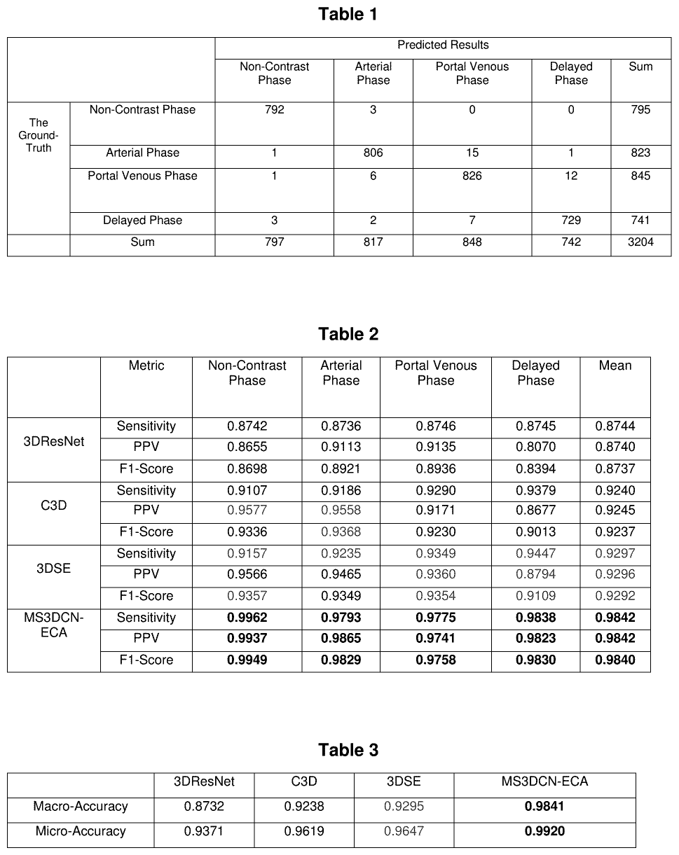

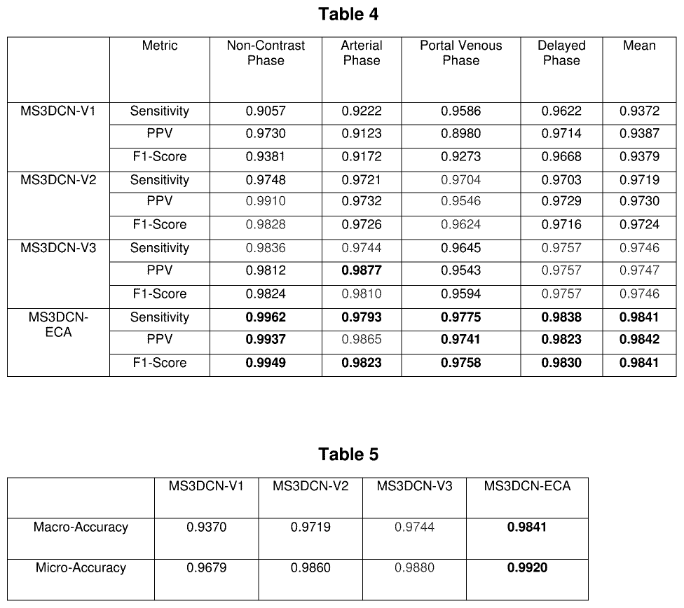

FIG. 5 provides a schematic diagram of an exemplary networked or distributed computing environment 1200. The distributed computing environment comprises computing objects 1210, 1212, etc. and computing objects or devices 1220, 1222, 1224, 1226, 1228, etc., which may include programs, methods, data stores, programmable logic, etc., as represented by applications 1230, 1232, 1234, 1236, 1238 and data store(s) 1240. It can be appreciated that computing objects 1210, 1212, etc. and computing objects or devices 1220, 1222, 1224, 1226, 1228, etc. may comprise different devices, including a multimedia display device or similar devices depicted within the illustrations, or other devices such as a mobile phone, personal digital assistant (PDA), audio/video device, MP3 players, personal computer, laptop, etc. It should be further appreciated that data store(s) 1240 can include one or more cache memories, one or more registers, or other similar data stores disclosed herein.

Each computing object 1210, 1212, etc. and computing objects or devices 1220, 1222, 1224, 1226, 1228, etc. can communicate with one or more other computing objects 1210, 1212, etc. and computing objects or devices 1220, 1222, 1224, 1226, 1228, etc. by way of the communications network 1242, either directly or indirectly. Even though illustrated as a single element in FIG. 5, communications network 1242 may comprise other computing objects and computing devices that provide services to the system of FIG. 5, and/or may represent multiple interconnected networks, which are not shown. Each computing object 1210, 1212, etc. or computing object or devices 1220, 1222, 1224, 1226, 1228, etc. can also contain an application, such as applications 1230, 1232, 1234, 1236, 1238, that might make use of an API, or other object, software, firmware and/or hardware, suitable for communication with or implementation of the techniques and disclosure described herein.

There are a variety of systems, components, and network configurations that support distributed computing environments. For example, computing systems can be connected together by wired or wireless systems, by local networks or widely distributed networks. Currently, many networks are coupled to the Internet, which provides an infrastructure for widely distributed computing and encompasses many different networks, though any network infrastructure can be used for exemplary communications made incident to the systems automatic diagnostic data collection as described in various embodiments herein.

Thus, a host of network topologies and network infrastructures, such as client/server, peer-to-peer, or hybrid architectures, can be utilized. The “client” is a member of a class or group that uses the services of another class or group to which it is not related. A client can be a process, i.e., roughly a set of instructions or tasks, which requests a service provided by another program or process. The client process utilizes the requested service, in some cases without having to “know” any working details about the other program or the service itself.

In a client/server architecture, particularly a networked system, a client is usually a computer that accesses shared network resources provided by another computer, e.g., a server. In the illustration of FIG. 5, as a non-limiting example, computing objects or devices 1220, 1222, 1224, 1226, 1228, etc. can be thought of as clients and computing objects 1210, 1212, etc. can be thought of as servers where computing objects 1210, 1212, etc., acting as servers provide data services, such as receiving data from client computing objects or devices 1220, 1222, 1224, 1226, 1228, etc., storing of data, processing of data, transmitting data to client computing objects or devices 1220, 1222, 1224, 1226, 1228, etc., although any computer can be considered a client, a server, or both, depending on the circumstances.

A server is typically a remote computer system accessible over a remote or local network, such as the Internet or wireless network infrastructures. The client process may be active in a first computer system, and the server process may be active in a second computer system, communicating with one another over a communications medium, thus providing distributed functionality and allowing multiple clients to take advantage of the information-gathering capabilities of the server. Any software objects utilized pursuant to the techniques described herein can be provided standalone, or distributed across multiple computing devices or objects.

In a network environment in which the communications network 1242 or bus is the Internet, for example, the computing objects 1210, 1212, etc. can be Web servers with which other computing objects or devices 1220, 1222, 1224, 1226, 1228, etc. communicate via any of a number of known protocols, such as the hypertext transfer protocol (HTTP). Computing objects 1210, 1212, etc. acting as servers may also serve as clients, e.g., computing objects or devices 1220, 1222, 1224, 1226, 1228, etc., as may be characteristic of a distributed computing environment.

Reference throughout this specification to “one embodiment,” “an embodiment,” “an example,” “an implementation,” “a disclosed aspect,” or “an aspect” means that a particular feature, structure, or characteristic described in connection with the embodiment, implementation, or aspect is included in at least one embodiment, implementation, or aspect of the present disclosure. Thus, the appearances of the phrase “in one embodiment,” “in one example,” “in one aspect,” “in an implementation,” or “in an embodiment,” in various places throughout this specification are not necessarily all referring to the same embodiment. Furthermore, the particular features, structures, or characteristics may be combined in any suitable manner in various disclosed embodiments.

As utilized herein, terms “component,” “system,” “architecture,” “engine” and the like are intended to refer to a computer or electronic-related entity, either hardware, a combination of hardware and software, software (e.g., in execution), or firmware. For example, a component can be one or more transistors, a memory cell, an arrangement of transistors or memory cells, a gate array, a programmable gate array, an application specific integrated circuit, a controller, a processor, a process running on the processor, an object, executable, program or application accessing or interfacing with semiconductor memory, a computer, or the like, or a suitable combination thereof. The component can include erasable programming (e.g., process instructions at least in part stored in erasable memory) or hard programming (e.g., process instructions burned into non-erasable memory at manufacture).

By way of illustration, both a process executed from memory and the processor can be a component. As another example, an architecture can include an arrangement of electronic hardware (e.g., parallel or serial transistors), processing instructions and a processor, which implement the processing instructions in a manner suitable to the arrangement of electronic hardware. In addition, an architecture can include a single component (e.g., a transistor, a gate array, . . . ) or an arrangement of components (e.g., a series or parallel arrangement of transistors, a gate array connected with program circuitry, power leads, electrical ground, input signal lines and output signal lines, and so on). A system can include one or more components as well as one or more architectures. One exemplary system can include a switching block architecture comprising crossed input/output lines and pass gate transistors, as well as power source(s), signal generator(s), communication bus(es), controllers, I/O interface, address registers, and so on. It is to be appreciated that some overlap in definitions is anticipated, and an architecture or a system can be a stand-alone component, or a component of another architecture, system, etc.

In addition to the foregoing, the disclosed subject matter can be implemented as a method, apparatus, or article of manufacture using typical manufacturing, programming or engineering techniques to produce hardware, firmware, software, or any suitable combination thereof to control an electronic device to implement the disclosed subject matter. The terms “apparatus” and “article of manufacture” where used herein are intended to encompass an electronic device, a semiconductor device, a computer, or a computer program accessible from any computer-readable device, carrier, or media. Computer-readable media can include hardware media, or software media. In addition, the media can include non-transitory media, or transport media. In one example, non-transitory media can include computer readable hardware media. Specific examples of computer readable hardware media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick, key drive . . . ). Computer-readable transport media can include carrier waves, or the like. Of course, those skilled in the art will recognize many modifications can be made to this configuration without departing from the scope or spirit of the disclosed subject matter.

Unless otherwise indicated in the examples and elsewhere in the specification and claims, all parts and percentages are by weight, all temperatures are in degrees Centigrade, and pressure is at or near atmospheric pressure.

### Experiments/Examples

First the data set used in the work and present comparative experiments between the method of the present invention and other competing methods is described. Then a previously conducted ablation study is reported upon, which verifies the effectiveness of the multi-scale information fusion and ECA.

### Data Set Description and Preprocessing

A total of 2714 CT scans 210 of FIG. 2, were collected from multiple hospitals. The numbers of four phases are 2677, 2693, 2714 and 2619 respectively. Not considered were thick-cut CT phase samples whose slice thickness is equal to or larger than 5 mm, resulting into 10680 CT phase samples. They were split into a training set and a testing set by 70% and 30%. Thus, the numbers of CT phase samples in the training set and the testing set were 7476 and 3204, respectively. In the training set, there are 1882, 1870, 1869 and 1878 samples belonging to non-contrast, arterial, portal venous and delayed phases, respectively; while in the testing set, there are 795, 823, 845 and 741 samples belonging to non-contrast, arterial, portal venous and delayed phases, respectively.

All the CT scans have a resolution of 512×512 with various numbers of slices. To reduce the hardware resource requirement, the resolution was resized to 256×256, and the number of slices was set to 128. There were significant differences in image intensity of the CT scans since they were acquired by different equipment and protocols. For example, image intensities of CT scans from PYN (identify) are in the interval [−2048, 2048] in terms of Hounsfield unit, image intensities of CT scans from HKU (Hong Kong University) are in the interval [−3023, 2137], while image intensities of CT scans from HKU_SZH (identify) are in the interval [−1024, 3071]. Thus, after resizing the resolution of the CT scans, truncate intensities to the internal [40, 400], followed by normalizing to the interval from 0 to 255.

### Quantitative Comparative Result

The detailed classification result of the MS3DCN-ECA on the above testing set is set forth in Table 1. It is observed from the table that the MS3DCN-ECA can identify most of the samples of four phases successfully. Particularly, MS3DCN-ECA performs the best in identifying non-contrast phase, only misclassifying three samples. In addition, MS3DCN-ECA performs equally well on the other three phases.

Then the MS3DCN-ECA of the present invention is compared with three conventional methods: 3DResNet, C3D, and 3DSE in terms of sensitivity, positive predictive value (PPV), F1-score at the phase-level as shown in Table 2, macro-accuracy and micro-accuracy of the overall performance as shown in Table 3. From Table 2, it can be seen that the MS3DCN-ECA described herein outperforms the second-best conventional method 3DSE by 5.46%, 5.45% and 5.48% in terms of mean sensitivity, mean PPV and mean F1-score respectively.

The advantages of MS3DCN-ECA over the conventional 3DSE methods is attributed to the facts that: 1) the former considers multi-scale information fusion to capture features for receptive fields of different sizes; and 2) the formers adopt ECA to learn cross-channel interaction effectively, instead of squeeze-and-extraction block (SE) in the latter. At the phase-level, MS3DCN-ECA achieves sensitivity of 0.9962 and PPV of 0.9937 on non-contrast phase, exceeding the second-best result 0.9157 and 0.9577 by 8.05% and 3.60%, respectively. The MS3DCN-ECA achieves sensitivity of 0.9793 and PPV of 0.9865 on the arterial phase, exceeding the second-best 0.9235 and 0.9558 by 5.58% and 3.07%, respectively. Further, the MS3DCN-ECA achieves sensitivity of 0.9775 and PPV of 0.9741 on venous phase, exceeding the second-best 0.9349 and 0.9360 by 4.26% and 3.81%, respectively. Finally, the MS3DCN-ECA achieves sensitivity of 0.9838 and PPV of 0.9823 on delayed phase, exceeding the second-best 0.9447 and 0.8794 by 3.91% and 10.29%, respectively.

From Table 3, it can be observed that MS3DCN-ECA achieved macro-accuracy of 0.9841 and micro-accuracy of 0.9920, which are better than the second-best results by 5.46% and 2.73% respectively. Overall, MS3DCN-ECA has clear superiority.

Thirdly, an ablation study was conducted to investigate the effects of multi-scale information fusion and ECA on performance improvement. Specifically, variants of the method described herein were denoted as MS3DCN-V1 and MS3DCN-V2 by disabling the branch whose input sizes are 256×256×128 and 128×128×128 respectively and deactivating ECA. MS3DCN-V3, which denotes the variant that deactivates ECA only. The results are shown in Tables 4-5.

As shown in Table 4, compared to MS3DCN-V1 and MS3DCN-V2, MS3DCN-V3 achieved better performance in terms of mean sensitivity, mean PPV and mean F1-score, which indicates the fusion of multi-scale information can bring performance improvement. Among them MS3DCN-ECA achieves better performance on non-contrast, portal venous and delayed phases, which indicates the effectiveness of ECA. Specifically, MS3DCN-ECA achieved sensitivity of 0.9962, PPV of 0.9937 and F1-score of 0.9949 on non-contrast phase, exceeding the second-best result by 1.26%, 0.27% and 1.13% respectively. MS3DCN-ECA achieves sensitivity of 0.9775, PPV of 0.9741 and F1-score of 0.9758 on portal venous phase, exceeding the second-best result by 0.71%, 1.95% and 1.34% respectively. MS3DCN-ECA achieves sensitivity of 0.9838, PPV of 0.9823 and F1-score of 0.9830 on delayed phase, exceeding the second-best result by 0.81%, 0.66% and 0.73% respectively.

From Table 5 it can be seen that MS3DCN-ECA performed better than MS3DCN-V3 by 0.97% and 0.40% in terms of macro-accuracy and micro-accuracy, respectively. Overall, the results from Tables 4-5 indicates multi-scale information fusion and ECA deliver performance improvement.

### Quantitative Comparative Result

Referring to FIG. 3, there are shown comparisons of CT phase and feature maps of images from the abdomen of three different patients. It can be observed that there are obvious differences across the phases corresponding to key regions of interests, which are used to identify CT phases. For example, the zone corresponding to arteries in the black solid-line box of the feature maps in FIG. 3 has little difference from that of the liver in the non-contrast phase, while there are significant differences between zones corresponding to arterial areas and zones corresponding to the liver in the arterial phase. In the portal venous phase, zones corresponding to liver veins or lesions in the black dash-line boxes in FIG. 3, show an increase in color toward red compared to the zones corresponding to the artery. In the delayed phase, zones of liver and artery show more blue and less yellow, compared to zones of the liver and artery in arterial and portal venous phases. Zones corresponding to veins in portal venous and delayed phases have more yellow and red than those in the non-contrast phase and arterial phases. Overall, zones corresponding to artery and liver veins in the feature maps have witnessed significant differences across the four phases.

With respect to any figure or numerical range for a given characteristic, a figure or a parameter from one range may be combined with another figure or a parameter from a different range for the same characteristic to generate a numerical range.

Other than in the operating examples, or where otherwise indicated, all numbers, values and/or expressions referring to quantities of ingredients, reaction conditions, etc., used in the specification and claims are to be understood as modified in all instances by the term “about.”

While the invention is explained in relation to certain embodiments, it is to be understood that various modifications thereof will become apparent to those skilled in the art upon reading the specification. Therefore, it is to be understood that the invention disclosed herein is intended to cover such modifications as fall within the scope of the appended claims.

1. A three dimensional classification system for recognizing cross-sectional images automatically, comprising:
a memory that stores computer executable components; and
a processor that executes the computer executable components stored in the memory, wherein the computer executable components comprise:
rescaling a plurality of cross-sectional images, and feeding the rescaled plurality of cross-sectional images into two branches;
feeding the rescaled plurality of cross-sectional images into a first branch for performing a plurality of convolutions on the rescaled plurality of cross-sectional images directly to learn features for distinguishing phases thereof;
feeding the rescaled plurality of cross-sectional images into a second branch for reducing resolution, then performing a plurality of convolutions on the reduced resolution plurality of cross-sectional images to learn features for distinguishing phases thereof; and
concatenating convolutional output channels from the two branches to fuse global and local features, on which two fully-connected layers are stacked as a classifier to recognize cross-sectional volumetric images accurately and quickly.

2. The three dimensional classification system of claim 1, wherein the cross-sectional images comprise computed tomography (CT) images.

3. The three dimensional classification system of claim 1, wherein the cross-sectional images comprise magnetic resonance images.

4. The three dimensional classification system of claim 1, wherein spatial relationships across slices of the plurality of images are analyzed.

5. The three dimensional classification system of claim 1, wherein the first branch performs four convolutions on the rescaled plurality of cross-sectional images.

6. The three dimensional classification system of claim 1, wherein the second branch performs four convolutions on the reduced resolution plurality of cross-sectional images.

7. The three dimensional classification system of claim 1 configured to facilitate an assessment of anatomical structures based upon a stereoscopic volumetric quantification.

8. A machine learning system comprising the three dimensional classification system of claim 1.

9. A method of recognizing cross-sectional images, comprising:
rescaling a plurality of cross-sectional images, and feeding the rescaled plurality of cross-sectional images into two branches;
feeding the rescaled plurality of cross-sectional images into a first branch for performing a plurality of convolutions on the rescaled plurality of cross-sectional images directly to learn features for distinguishing phases;
feeding the rescaled plurality of cross-sectional images into a second branch for reducing resolution, then performing a plurality of convolutions on the reduced resolution plurality of cross-sectional images to learn features for distinguishing phases; and
concatenating convolutional output channels from the two branches to fuse global and local features, on which two fully-connected layers are stacked as a classifier to recognize cross-sectional volumetric images accurately and quickly.

10. The method of recognizing cross-sectional images of claim 9, wherein the first branch performs four convolutions on the rescaled plurality of cross-sectional images.

11. The method of recognizing cross-sectional images of claim 9, wherein the second branch performs four convolutions on the reduced resolution plurality of cross-sectional images.

12. The method of recognizing cross-sectional images of claim 9, further comprising:
facilitating an assessment of anatomical structures based upon a stereoscopic volumetric quantification.

13. A method of diagnosing cancer comprising using the method of recognizing cross-sectional images of claim 9.

14. A non-transitory machine-readable storage medium, comprising executable instructions that, when executed by a processor, facilitate performance of operations, comprising:
rescaling a plurality of cross-sectional images, and feeding the rescaled plurality of cross-sectional images into two branches;
feeding the rescaled plurality of cross-sectional images into a first branch for performing a plurality of convolutions on the rescaled plurality of cross-sectional images directly to learn features for distinguishing phases;
feeding the rescaled plurality of cross-sectional images into a second branch for reducing resolution, then performing a plurality of convolutions on the reduced resolution plurality of cross-sectional images to learn features for distinguishing phases; and
concatenating convolutional output channels from the two branches to fuse global and local features, on which two fully-connected layers are stacked as a classifier to recognize cross-sectional volumetric images accurately and quickly.
